(19) United States
(12) Patent Application Publication
(10) Pub. No.: US 2005/0079509 A1
(43) Pub. Date: Apr. 14, 2005

(54) Methods for identifying suitable nucleic acid normalization probe sequences for use in nucleic acid arrays

(76) Inventor: Karen W. Shannon, Los Gatos, CA (US)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/686,092

(22) Filed: Oct. 14, 2003

(51) Int. Cl. C12Q 1/68; G06F 19/00; G01N 33/48; G01N 33/50

(52) U.S. Cl. 435/6; 702/20

(57) ABSTRACT

Methods of identifying a sequence of a probe, e.g., a biopolymeric probe, such as a nucleic acid, that is suitable for use as a surface immobilized normalization probe on a nucleic acid array are provided. A feature of the subject methods is that a set of computationally determined initial candidate sequences are empirically evaluated to obtain functional data that is then employed to evaluate the candidate sequences for suitability as normalization probes. Sequences identified as suitable for use as normalization probes according to the subject methods are ones that do not cluster with other probes of the candidate set, exhibit high signal intensity and exhibit substantially no differential expression across a large number of samples. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

### FIELD OF THE INVENTION

The field of this invention is nucleic acid arrays.

### BACKGROUND OF THE INVENTION

Arrays of binding agents or probes, such as polypeptide and nucleic acids, have become an increasingly important tool in the biotechnology industry and related fields. These binding agent arrays, in which a plurality of probes are positioned on a solid support surface in the form of an array or pattern, find use in a variety of different fields, e.g., genomics (in sequencing by hybridization, SNP detection, differential gene expression analysis, identification of novel genes, gene mapping, finger printing, etc.) and proteomics.

In using such arrays, the surface bound probes are contacted with molecules or analytes of interest, i.e., targets, in a sample. Targets in the sample bind to the complementary probes on the substrate to form a binding complex. The pattern of binding of the targets to the probe features or spots on the substrate produces a pattern on the surface of the substrate and provides desired information about the sample. In most instances, the targets are labeled with a detectable label or reporter such as a fluorescent label, chemiluminescent label or radioactive label. The resultant binding interaction or complexes of binding pairs are then detected and read or interrogated, for example by optical means, although other methods may also be used depending on the detectable label employed. For example, laser light may be used to excite fluorescent labels bound to a target, generating a signal only in those spots on the substrate that have a target, and thus a fluorescent label, bound to a probe molecule. This pattern may then be digitally scanned for computer analysis.

Normalization is a general problem in the analysis of data for nucleic acid microarrays hybridized to samples labeled in 2 or more colors. Normalization is the process by which the data from all color channels is brought onto the same relative scale. Such resealing is a prerequisite to the calculation of different expression ratios because if the data are not on the same relative scale, the calculated expression ratios produced from the data will be mutliplied by some unknown factor or function.

Current methods of normalization generally rely on two steps. The first step is to identify a subset of data for the expression ratio that (at least on average) is believed to be known. For example, one can employ a set of “housekeeping genes” (genes believed to be uniformly expressed in different sample types) or one can use all statistically significant data (if the the number of differentially expressed genes is believed to be small compared to the total population). The second step is to rescale the data channels according to a suitable model. The model may be as simple as division of all data in each channel by the arithmetic or geometric mean of the data in that channel, or as complex as fitting to a non-linear function.

The above methods rely on the identification of a subset of the data for use as normalization probes. These probes can be identified a priori, as in the use of housekeeping genes, or they can be identified as part of the normalization process, as is done using the Longest Order-Preserving Set (LOPS) method or using the Rank Order Normalization protocol (Agilent Technologies, Palo Alto, Calif.). Housekeeping gene sets suffer from the difficulty that, upon detailed examination, many such sets turn out not to be uniformly expressed, i.e., differentially expressed, across different samples and thus are generally not useful across a wide variety of sample sets. Methods that rely on “in process” identification of normalization probes may fail if the number of differentially expressed genes is not small compared to the total population, or if the total population is small.

As such, there is continued interest in the identification of normalization probes for using in nucleic acid array assays.

Relevant Literature

U.S. patents of interest include: 6,591,196; 6,251,588 and 5,556,749. Published U.S. patent applications of interest include: 20030156136 and 20030065449.

### SUMMARY OF THE INVENTION

Methods of identifying a sequence of a probe, e.g., a biopolymeric probe, such as a nucleic acid, that is suitable for use as a surface immobilized normalization probe on a nucleic acid array are provided. A feature of the subject methods is that a set of computationally determined initial candidate sequences are empirically evaluated to obtain functional data that is then employed to evaluate the candidate sequences for suitability as normalization probes. Sequences identified as suitable for use as normalization probes according to the subject methods are ones that do not cluster with other probes of the candidate set, exhibit high signal intensity and exhibit substantially no differential expression across a large number of samples. The subject invention also includes algorithms for performing the subject methods recorded on a computer readable medium, as well as computational analysis systems that include the same. Also provided are nucleic acid arrays produced with normalization probes having sequences identified by the subject methods, as well as methods for using the same.

### BRIEF DESCRIPTIONS OF THE DRAWING

FIG. 1 shows a flowchart representing the steps of the subject methods.

FIG. 2 provides a graph of log ratio vs. signal intensity for a series of normalization probes identified according to an embodiment of the subject invention, as described in the Experimental Section, below.

### DEFINITIONS

In the present application, unless a contrary intention appears, the following terms refer to the indicated characteristics.

Unless defined otherwise, all technical and scientific terms used herein have the same meaning as commonly understood by one of ordinary skill in the art to which this invention belongs. Still, certain elements are defined below for the sake of clarity and ease of reference.

A “biopolymer” is a polymer of one or more types of repeating units. Biopolymers are typically found in biological systems and particularly include polysaccharides (such as carbohydrates), peptides (which term is used to include polypeptides and proteins) and polynucleotides as well as their analogs such as those compounds composed of or containing amino acid analogs or non-amino acid groups, or nucleotide analogs or non-nucleotide groups. Biopolymers include polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. Biopolymers include DNA (including cDNA), RNA, oligonucleotides, and PNA and other polynucleotides as described in U.S. Pat. No. 5,948,902 and references cited therein (all of which are also incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (e.g., a single amino acid or nucleotide with two linking groups one or both of which may have removable protecting groups).

An “array,” includes any one-dimensional, two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, light directed synthesis fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein. Also, instead of drop deposition methods, light directed fabrication methods may be used, as are known in the art. Interfeature areas need not be present particularly when the arrays are made by light directed synthesis protocols.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exist intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic and other materials are also suitable.

The term “flexible” is used herein to refer to a structure, e.g., a bottom surface or a cover, that is capable of being bent, folded or similarly manipulated without breakage. For example, a cover is flexible if it is capable of being peeled away from the bottom surface without breakage.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

The substrate may be flexible (such as a flexible web). When the substrate is flexible, it may be of various lengths including at least 1 m, at least 2 m, or at least 5 m (or even at least 10 m).

The term “rigid” is used herein to refer to a structure e.g., a bottom surface or a cover that does not readily bend without breakage, i.e., the structure is not flexible.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

The term “stringent conditions” refers to conditions under which a probe will hybridize preferentially to its target subsequence, and to a lesser extent to, or not at all to, other sequences. Put another way, the term “stringent hybridization conditions” as used herein refers to conditions that are compatible to produce duplexes on an array surface between complementary binding members, e.g., between probes and complementary targets in a sample, e.g., duplexes of nucleic acid probes, such as DNA probes, and their corresponding nucleic acid targets that are present in the sample, e.g., their corresponding mRNA analytes present in the sample. A “stringent hybridization” and “stringent hybridization wash conditions” in the context of nucleic acid hybridization (e.g., as in array, Southern or Northern hybridizations) are sequence dependent, and are different under different environmental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 nmM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a probe. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C. In instances wherein the nucleic acid molecules are deoxyoligonucleotides (“oligos”), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed, as appropriate.

By “remote location,” it is meant a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. An array “package” may be the array plus only a substrate on which the array is deposited, although the package may include other features (such as a housing with a chamber). A “chamber” references an enclosed volume (although a chamber may be accessible through one or more ports). It will also be appreciated that throughout the present application, that words such as “top,” “upper,” and “lower” are used in a relative sense only.

A “computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

A “processor” references any hardware and/or software combination that will perform the functions required of it. For example, any processor herein may be a programmable digital microprocessor such as available in the form of a electronic controller, mainframe, server or personal computer (desktop or portable). Where the processor is programmable, suitable programming can be communicated from a remote location to the processor, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

### DETAILED DESCRIPTION OF THE INVENTION

Methods of identifying a sequence of a probe, e.g., a biopolymeric probe, such as a nucleic acid, that is suitable for use as a surface immobilized normalization probe on a nucleic acid array are provided. A feature of the subject methods is that a set of computationally determined initial candidate sequences are empirically evaluated to obtain functional data that is then employed to evaluate the candidate sequences for suitability as normalization probes. Sequences identified as suitable for use as normalization probes according to the subject methods are ones that do not cluster with other probes of the candidate set, exhibit high signal intensity and exhibit substantially no differential expression across a large number of different samples. The subject invention also includes algorithms for performing the subject methods recorded on a computer readable medium, as well as computational analysis systems that include the same. Also provided are nucleic acid arrays produced with probes having sequences identified by the subject methods, as well as methods for using the same.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described. Methods recited herein may be carried out in any order of the recited events which is logically possible, as well as the recited order of events.

All patents and other references cited in this application, are incorporated into this application by reference except insofar as they may conflict with those of the present application (in which case the present application prevails).

As summarized above, the subject invention provides methods of identifying or designing normalization probes for use in an array structures, where the normalization probes are chemical probes, e.g., biopolymeric probes, such as nucleic acids. While the following description is provided in terms of normalization nucleic acid probe design protocols for ease and clarity of description, the scope of the invention is not so limited, but instead extends to the identification or design of suitable normalization probes for use in any type of array structure.

In further describing the subject invention, the methods for identifying suitable normalization probe sequences are described first in greater detail, followed by a review of arrays that may be produced using probes identified by the subject methods as well as representative applications for such arrays.

Methods

As summarized above, the subject invention provides methods of identifying a sequence of a nucleic acid that is suitable for use as a surface immobilized normalization probe for a nucleic acid array. In other words, the subject invention provides methods of designing nucleic acid probes that are suitable for use as normalization probes on nucleic acid arrays. The subject methods result in the identification of normalization probes that exhibit high signal intensity and little, if any, differential expression across a plurality of different types of samples. A feature of the subject methods is that they include both computational steps and empirical steps, where specifically a collection of candidate probe sequences for a given target nucleic acid are first computationally identified from the sequence of the target nucleic acid of interest, where the initially identified candidate sequences are subsequently tested empirically and then further evaluated using additional computational steps in order to identify one or more suitable normalization probes.

In many embodiments, the subject methods include the following steps:
(a) identifying a plurality of candidate probe sequences for the target nucleic acid;
(b) empirically evaluating each of the identified candidate probe sequences;
(c) clustering the identified candidate probe sequences into two or more groups of candidate probe sequences based on observed empirical data values, where clustered members exhibit substantially the same performance across a plurality of different experimental sets, specifically a plurality of differential gene expression experiments; and
(d) evaluating any remaining non-clustered candidate probe sequences for those that exhibit high signal intensity and substantially no differential expression accross a plurality of different samples to identify sequences suitable for use in normalization probes.

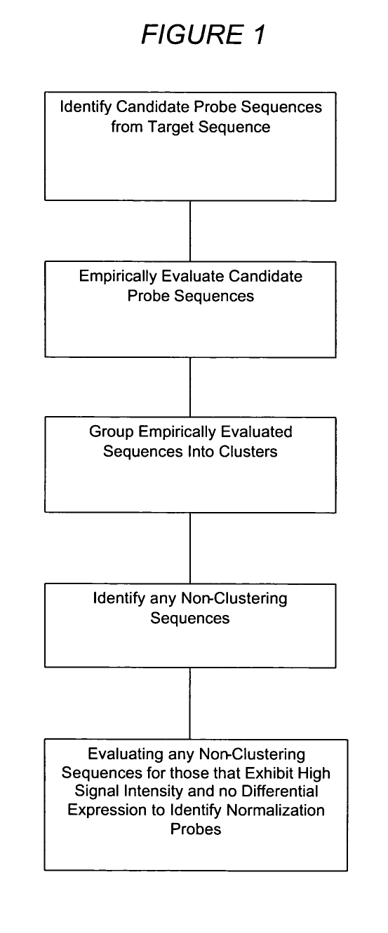

FIG. 1 provides a flow chart showing each of the above steps of the subject methods. In further describing the subject methods, each of the above steps is now reviewed separately in greater detail below.

Candidate Probe Identification

As mentioned above, the first step in the subject methods is to identify a plurality of candidate probe sequences for a given target nucleic acid of interest. The target nucleic acid of interest is generally a nucleic acid of known sequence, where the length of the nucleic acid may vary, but typically ranges from about 200 nt to about 4,000 nt, such as from about 400 nt to about 2,500 nt, including from about 800 nt to about 1,500 nt. In many embodiments, the target nucleic acid has the sequence of an mRNA transcript of interest or the complementary sequence thereof, or the sequence of a first or second strand DNA prepared from an mRNA of interest.

The candidate probes are identified based on a least one selection criterion, wherein in many embodiments a plurality of different selection criteria are employed together to identify the candidate probes from the target nucleic acid sequence, where by plurality is meant at least about 2, and plurality may be as greater as 10 or more, but is typically less than 5, e.g., 2 to 3.

One selection criterion of interest that may be employed is distance from the 3′-end of the mRNA transcript that corresponds to the target nucleic acid, e.g., that is the target nucleic acid or is the complement of the target nucleic acid, or from which the target nucleic acid is derived, e.g., where the target nucleic acid is first or second strand cDNA. When this criterion is employed, candidate sequences of the target nucleic acid are chosen that are within at least about 2,000 nt, usually within about 1,500 nt and more usually within about 800 nt of the 3′ end of the mRNA that corresponds to the target nucleic acid.

Another selection criterion of interest is the base composition of the probe sequence. When this criterion is employed, sequences that are abnormally GC rich or poor, long runs of a single base, and/or base compositions that are known to generate unacceptable array features, e.g., under in situ production conditions are avoided. Sequences that are abnormally GC rich or poor are those sequences whose number % of G and C bases are greater than about 30, such as greater than about 35, or less than about 60, such as less than about 45. By “long run” of a single base is meant a stretch of nucleotides of the same base that is greater than about 6, such as greater than about 10. Sequences that are known to generate unacceptable array features include, but are not limited to those containing stretches of at least 10 Gs.

Another selection criterion of interest is homology of the candidate probe sequence to other sequences from the same organism, i.e., to other mRNA transcripts or complements thereof of the same organism from which the target sequence of interest for which the probe is being designed is obtained. Sequences with a high potential to hybridize to more than one mRNA transcript from a given organism are avoided. Cross-hybridization potential of candidate sequences may be estimated via thermodynamic scoring of the output of BLAST, a standard bioinformatics application used to detect sequence homology and well known to those of skill in the art, or any other convenient cross-hybridization potential assessment protocol. Use of this criterion results in the identification of probe sequences that are specific for the target nucleic acid of interest.

In certain embodiments, the identification process or algorithm that is employed is one in which parameters are used that minimize the number of identified candidate probe sequences that overlap with each other. Any of the above listed criteria may be adjusted in order to result in minimal overlap of the identified candidate probe sequences. The overlap parameter is designed to yield candidate probes that span the target—if it is not specified, the algorithm employed, may identify probes that are heavily overlapped (up to 59 out of 60 bases). While these may be the best probes, using such a set of candidates confounds the clustering analysis, since almost by definition such probes will cluster tightly.

Using the above protocol, a plurality of candidate probe sequences are identified for a given target nucleic acid. In many embodiments, the number of identified candidate probe nucleic acid sequences is at least about 5, usually at least about 7 and may be as great as 15, 20 or more, but typically does not exceed about 15, where in certain embodiments, the number of candidate probe sequences identified for a given target nucleic acid ranges from about 7 to 12, e.g., 8, 9, 10 or 11.

In certain embodiments, an algorithm is employed, e.g., in conjunction with a computational analysis system, to identify candidate probe sequences from a target nucleic acid. Any convenient algorithm or process capable of performing the above function may be employed. Of interest in many embodiments are the Agilent probe design algorithms (Agilent Technologies, Palo Alto, Calif.), where the algorithms are employed in identification of candidate probe sequences. Specifically, the design parameters that may be employed include: 1) the preferred and allowed distances from the 3′ end, 2) the number of probes required before ending base composition iteration (where a suitable number typically ranges from about 20 to about 200, usually from about 50 to about 100), 3) the criteria used to label probes as “overlap” (where “overlap” may be defined as probes whose sequences overlap by a number of bases, for example greater than 10 nt, more typically greater than 40 nt), and 4) the number of probes required before the homology calculation (where a suitable number typically ranges from about 10 to about 40, usually from about 12 to about 20).

Another algorithm of interest includes the probe selection algorith described in pending U.S. application Ser. No. 09/659,173; the disclosure of which is herein incorporated by reference.

As indicated above, the above first step in the subject methods results in the identification of a plurality of different candidate probe sequences for a given target nucleic acid.

Empirical Evaluation of Identified Candidate Probe Nucleic Acid Sequences

In the next step of the subject methods, each of the identified candidate probe sequences is evaluated empirically. Specifically, each of the identified candidate probe sequences is evaluated for its performance under a plurality of different experimental sets, specifically a plurality of differential gene expression experiments to obtain a collection of empirically obtained performance data values for each of the candidate nucleic acid probe sequences for each of the plurality of different experimental conditions. In many embodiments, the experimental conditions are differential gene expression assay experiments, where a given experimental condition is a differential gene expression assay using a particular nucleic acid sample pair, where each sample of the pair is obtained from a different source, e.g., tissue or cell line. Differential gene expression array-based assays are well known to those of skill in the art. The number of different differential gene expression array based assays for which a given candidate probe is empirically evaluated may vary, where the number may range from about 2 to about 20, such as from about 5 to about 15, including from about 7 to about 12, e.g., 10. Any two differential gene expression assays or protocols are considered different if at least one of the nucleic acid samples making up the pairs of any two pairs differs between the two pairs.

The differential gene expression assays are typically performed by first providing an array of candidate nucleic acid probes immobilized on a surface of a solid support, where the array includes a substrate surface immobilized nucleic acid candidate probe for each of the identified candidate probe sequences to be empirically evaluated. In other words, an array is provided that includes a probe for each of the to be evaluated candidate probe sequences, i.e., all of the to be evaluated candidate probe sequences have corresponding probes on the array that include the same sequence. The arrays of candidate probes may be provided in a number of different ways, e.g., via in situ production, as described in U.S. Pat. Nos. 6,451,998; 6,446,682; 6,440,669; 6,420,180; 6,372,483; 6,323,043; and 6,242,266; the disclosures of which patents are herein incorporated by reference.

The surface immobilized candidate probes having the sequences of the candidate probe sequences are then contacted with two or more sets of nucleic acid sample pairs under differential gene expression analysis conditions to evaluate the probes. In certain embodiments, an identical candidate probe array is contacted with each different sample pair of the set of different sample pairs, while in other embodiments, the same nucleic acid array may be contacted with two or more sample pairs, so long as any hybridized targets from any previous assay are efficiently removed or “stripped” prior to contact with the next sample pair. Differential gene expression assay protocols are further described below.

In a representative example of the above empirical evaluation step of the subject methods, multiple copies of a microarray that includes candidate 60-mer probes having sequences identified by the prior sequence identification step are produced using an in situ nucleic acid array synthesis protocol. These resultant microarrays are then hybridized to 10 different tissue/cell line combinations (4 replicates per sample pair): one self-vs-self and 9 sample pairs chosen to maximize the number of mRNAs that are differentially expressed between the members of the pair. The arrays are then scanned, as described in greater detail below, and the feature data are extracted using extraction software, such as Agilent's Feature Extraction software (available from Agilent Technologies, Palo Alto, Calif.). Where desired, the resultant data may be placed in tabular form or collated into a relational database or otherwise organized. Typically, the feature extraction protocol computes P-values, specifically the likelihood that the P-value is significantly different from 0. The feature data are further processed to exclude data from features that do not satisfy certain quality control measures, e.g., signal saturation or the presence of too many outlier pixel values and to exclude data from probes that do not generate sufficient signal in any of the experiments. The obtained feature data are further processed by combining replicate experiments using statistical weights derived from the P-values associated with each feature, e.g., by using a processing algorithm designed for this purpose.

The above empirical evaluation process results in the production of a collection of empirically obtained data values for each candidate probe sequence, where the empirical data values are measures of performance across a plurality of different experimental sets, specifically a plurality of differential gene expression experiments. Specifically, a collection of probe performance data values (e.g., in the form of log ratio values) for each different differential gene expression experiment is obtained for each candidate probe, such that for each probe one obtains an empirical or experimentally determined measure of that probe's performance in each of a number of different differential gene expression assays, e.g., a value is obtained to represent performance of each probe in each experiment. The data making up a given collection of data values may be raw data or processed, and may be a measure of hybridization efficiency, signal intensity, signal ratio, signal log-ratio or combination thereof.

Clustering Candidate Probe Sequences

In the next step of the subject methods, the candidate probe sequences are clustered into two or more groups of candidate probe sequences, where the candidate probe sequences are divided into two or more groups of candidate probe sequences based on the observed empirical data values obtained in the prior empirical evaluation step.

In many embodiments of this clustering or grouping step, one first obtains an expression vector for each of the candidate probe sequences using the candidate probe sequence's collection of empirical data values. From the obtained expression vector for each candidate probe sequence, one then derives a similarity matrix for the set of the candidate probe sequences, where the similarity matrix provides a measure of how similar the candidate probe sequence functions as compared to the other candidate probe sequences being evaluated. Based on the derived similarity matrix for the set of candidate probe sequences, the candidate probe sequences are then grouped into two or more groups. Each of the above substeps of the clustering step is now reviewed separately in greater detail.

As indicated above, the first substep of the clustering step is the generation of an expression vector for each candidate probe sequence, where the expression vector is generated using the empirical data for the candidate probe sequence obtained in the empirical evaluation step described above. In many embodiments, the empirical data employed in the generation of the expression vector are the log ratio values from the sample-pair experiments, as indicated above. Where present, replicate log ratio values may be combined using error-weighted averaging. The combined log ratio data for candidate probes designed to target a single gene are used to populate an expression matrix I, where Iᵢⱼ is the measured expression level of probe i in experiment (condition) j. The number of columns in the expression matrix is the number of experiments performed for empirical validation, the number of rows in the expression matrix is the number of candidate probes designed to target a single gene. The significance of the similarity measure used depends on the number of experimental conditions performed. When Pearson correlation is used to measure the similarity of probes, the expression matrix should consist of at least 4 experiments, preferably 8 experiments, and even more preferably of at least 12 experiments. The matrix contains only data that survive the processing steps described above. As indicated above, certain feature data may be excluded, leading to missing values in the expression matrix, typically indicated by entering a special value (one that could never arise from an experiment, for example a log ratio of 10\hat{ }6) into the matrix. Subsequent processing steps must be able to process such a matrix.

In the next substep, a similarity matrix is derived or calculated from the obtained expression matrix of the first substep. In this similarity matrix, the entry Sᵢⱼ represents the similarity of the expression vectors for probes i and j. The similarity measure used for this step is independent of the clustering mechanism. Specific examples are Pearson's correlation coefficient (as described in Duda, R. O., and Hart, P. E. (1973). Pattern Classification and Scene Analysis. New York, John Wiley and Sons.), Kendall's rank correlation (as described in Kendall, M. G. (1970). Rank Correlation Methods (4^{th} edition). Griffin and Co. Ltd.), similarity measure based on the Euclidian distance, and weighted Pearson 's correlation.

Specific details on the above are provided below:

Let P be the expression matrix with m rows and n columns. Entry Pᵢⱼ of this matrix is the expression level of probe i in the experiment j. The entry Sᵢⱼ of similarity matrix S is the similarity between probe i and probe j, specific examples of how the similarity may be computed are given below.

1. Pearson's Correlation.
Duda, R. O., and Hart, P. E. (1973). Pattern Classification and Scene Analysis. New York, John Wiley and Sons.

Pearson's correlation Sᵢⱼ between probes i and j is

$${S}_{ij} = \frac{{{\sum }^{n}}_{k = 1} ( {P}_{ik} - {P}_{i} ) ( {P}_{jk} - {P}_{j} )}{{σ}_{i} {σ}_{j}} , where \\ {P}_{i} = \frac{1}{n} {{\sum }^{n}}_{k = 1} {P}_{ik} , \\ {σ}_{i}^{2} = {{\sum }^{n}}_{k = 1} {( {P}_{ik} - {P}_{i} )}^{2} .$$

2. Kendall's Rank Correlation.
Kendall, M. G. (1970). Rank Correlation Methods (4^{th} edition). Griffin and Co. Ltd.

3. Euclidean Distance Converted to Similarity Measure.

$${E}_{ij} = \sqrt{{{\sum }^{n}}_{k = 1} {( {P}_{ik} - {P}_{jk} )}^{2}}$$

Let min

$$E = {min}_{\begin{matrix}1 \leq i \leq m \\ 1 \leq j \leq n\end{matrix}} {E}_{ij} and max E = {max}_{\begin{matrix}1 \leq i \leq m \\ 1 \leq j \leq n\end{matrix}} {E}_{ij}$$

Then

$${S}_{ij} = 1 - \frac{{E}_{ij} - min E}{max E - min E} .$$

4. Weighted Pearson's Correlation.
Analogous to Pearson's correlation, but each experiment j is taken with weight wⱼ.
Given n weights w₁, W₂, . . . , Wₙ, such that

$${{\sum }_{j = 1}}^{n} {w}_{j} = 1 ,$$
weighted Pearson's correlation is computed in the following way:

$${S}_{ij} = \frac{{{\sum }^{n}}_{k = 1} ( {w}_{k} {P}_{ik} - {P}_{i} ) ( {w}_{k} {P}_{jk} - {P}_{j} )}{{σ}_{i} {σ}_{j}} ,$$
where Pᵢ and σᵢ are weighted mean and standard deviation of the probe i:

$${P}_{i} = \frac{1}{n} {{\sum }^{n}}_{k = 1} {w}_{k} {P}_{ik} , \\ {σ}_{i}^{2} = \frac{1}{n} {{\sum }^{n}}_{k = 1} {( {w}_{k} {P}_{ik} - {P}_{i} )}^{2} .$$

In the third substep, the candidate probes are clustered into one or more groups based on their similarity indices or matrices, as determined in the previous substep. In other words, the candidate probe sequences are placed into groups based on similar expression patterns. In this substep, a clustering algorithm is typically employed. Several clustering approaches can be applied here, where certain embodiments use the following approach. The input to the algorithm is a pair (S,t) where S is a n-by-n similarity matrix (n is equal to the number of candidate probes and ranges from about 3 to about 20, usually from about 5 to about 12) and t is a user-specified affinity threshold that determines what affinity level is considered significant (where t often ranges from about 0.3 to about 0.9, such as from about 0.5 to about 0.8). The algorithm constructs clusters incrementally and uses average inter-cluster similarity (affinity) between unassigned vertices and the current cluster to make its next decision to add or remove elements from groups. The clusters are “stable” when the average similarity exceeds the affinity threshold (t). In many embodiments, the algorithm allows input of up to 5 t values and iteratively performs the cluster analysis at decreasing affinity thresholds until a cluster of a user-defined minimum size is formed. Cluster members are assigned for each cluster and a cluster size and a cluster quality score is calculated. The quality score of a cluster is a measure of the likelihood of such a cluster occurring if data from unrelated probes from the data set were clustered. Highly unlikely clusters (i.e., those where the data cluster much more tightly than would be expected from data randomly selected according to the distribution of similarity between all probes in the data) are given high scores.

The above clustering protocol and substeps thereof (including the specific representative clustering protocol above that includes affinity value and scoring features) may be performed using any convenient algorithm. Of interest are algorithms that automate the steps of data filtering, data combination, clustering, cluster filtering, and probe selection, e.g., by performing all of the above described substeps. Of particular interest are algorithms that form a non-hierarchical clustering (i.e., the clusters are unrelated and cluster boundaries are determined by the algorithm) and do not assume a given number of clusters (i.e., the number of clusters is determined by the algorithm instead of being a constant given as an input parameter). In certain embodiments, the algorithm employed in this step is a CAST (Cluster Affinity Search Technique) clustering algorithm, as known to those of skill in the art and described in U.S. Pat. No. 6,421,668; the disclosure of which is herein incorporated by reference. See also U.S. Pat. No. 6,468,476, the disclosure of which is herein incorporated by reference, which further discloses Clustering programs or algorithms that may find use in the subject methods.

The above substep results in clustering or grouping of the different candidate probe sequences into two or more groups or clusters of sequences, where each cluster is made up of probe sequences that hybridize to a single target and behave similarly in gene expression experiments, both within a single experimental sample pair and across multiple sample experimental pairs.

The above substep may also provide one or more non-clustered candidate probe sequences, i.e., one or more sequences that do not cluster or group with any other sequences during the clustering step. Any resultant non-clustering candidate probe sequences are then evaulated in the next step of the subject methods for their suitability as normalization probes. Note that if no non-clustering probes are present following the clustering step, the following evaluation step is not performed. Instead, a new set of candidate probe sequences is generated, e.g., to a different target, and processed as described above, until one or more non-clustering sequences are identified that can be evaulated pursuant to the next step for their suitable as normalization probes.

Evaluation of Non-Clustering Probe Sequences for Suitability as Normalization Probes

In the next step of the subject methods, any remaining non-clustered candidate probe sequences, i.e., any candidate probe sequences that are not grouped with at least one other candidate probe sequence in the prior clustering step because they lack sufficient hybridization similarities, are evaluated for their suitability for use as normalization probes in an array-based assay. Specifically, any resultant non-clustering probe sequences are assessed for specific normalization probe suitability parameters. Specific representative normalization probe suitability parameters or criteria for which any resultant non-clustering probes are screened in this final step of the subject methods are: (a) signal intensity; and (b) variance in expression across multiple samples.

As such, in this evaluation step of the subject methods, the signal intensity provided by any resultant non-clustering candidate probes in at least one sample contact protocol, generally in at least one differential gene expression assay, and more typically in the plurality of different experimental sets described above, is assessed to determine whether the signal intensity of the probe in at least one of the test conditions, usually the average signal intensity of at least some of the test conditions and in certain embodiments the average signal intensity in all of the purality of test conditions, satisfies or meets a predetermined signal intensity threshold. In certain embodiments, the signal intensity threshold is at least about 2-fold, such as at least about 5-fold or more (e.g., 10-fold, 25-fold, 50-fold or more) instense than background. Typically, the signal intensity threshold is selected so that candidate probe sequences satisying or meeting the threshold exhibit a high signal intensity when used in an assay of a plurality of different nucleic acid samples.

In addition, any resultant non-clustering candidate probe sequences are evaluated in this step of the subject methods to determine whether they exhibit substantially no, if any, signal variation when employed in array-based assays of different nucleic acid samples performed according to the protocol described in the Experimental Section below. In other words, any resultant non-clustering candidate probe sequences are evaulated to identify those sequences that do not exhibit differential expression when exposed to a plurality of different nucleic acid samples. Stated another way, any candidate non-clustering sequences are evaulated to determine whether they provide a substantially uniform signal across a plurality of different samples. A given candidate probe sequence is considered to exhibit or provide substantially no, if any, signal variation if the mean log ratio of the signal provided by the probe accross the plurality of different samples is not significantly different from zero, where by “not significantly different from zero” means that the log ratio is between about 0.5 and −0.5, such as between about 0.4 and −0.4. Methods of determining the log ratio of a probe accross a number of different samples are well known by those of skill in the art, as further described in the Experimental section below, where representative megthods are also described in U.S. Pat. No. 6,591,196, as well as published U.S. Patent applications 20030156136 and 20030065449; the dislcosures of which are herein incorporated by reference. See also Baggerly et al., J Comput Biol. 2001;8(6):639-59. This substep of the subject methods yields candidate probe sequences that exhibit substantially no signal variation, i.e., substantially uniform signal, in otherwise identical array-based assays that differ from each other solely with respect to nucleic acid source. In many embodiments, the above-described uniformity of signal is observed accross at least about 5 different samples, such as at least about 10 different samples, including at least about 15 different samples.

The above-described evaulation step results in the identification of candidate probe sequences (if any) that are suitable for use as normalization probes on nucleic acid arrays. The identified candidate probe sequences are suitable for use as normalization probes because they are uniformly expressed with a known expression ratio (e.g., log ratio=0) across a plurality of sample types, where the sample types may be highly divergent. The above step distinguishes probes that do not show differential expression because the target is not present (which probes are not likely to be useful as normalization probes) and probes that do not show differential expression because they show low affinity hybridization to a multitude of targets (which probes are likely to be useful as targets).

In many embodiments, the normalization probe nucleic acid sequences identified using the subject methods are provided in text format or as a string of text, where the text represents or corresponds to the sequence of nucleotides of a probe nucleic acid. The nucleic acid sequences can be of any length, where the nucleic acid sequences are typically about 20 nt to about 100 nt in length, e.g., from about 20 to about 80 nt in length, e.g., 25 nt, 60 nt, etc. However, nucleic acid sequences of lesser or greater length may be identified as appropriate. Suitable nucleic acid normalization probes produced therefrom may be oligonucleotides or polynucleotides, as will be described in greater detail below.

One or more aspects of the above methodology may be in the form of computer readable media having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically, optically or by other means. Accordingly, stored programming embodying steps for carrying-out the subject methods may be transferred to a computer such as a personal computer (PC), (i.e., accessible by a researcher or the like), by physical transfer of a CD, floppy disk, or like medium, or may be transferred using a computer network, server, or other interface connection, e.g., the Internet.

In one embodiment of the subject invention, a system of the invention may include a single computer or the like with a stored algorithm capable of carrying out suitable probe identification methods, i.e., a computational analysis system. In certain embodiments, the system is further characterized in that it provides a user interface, where the user interface presents to a user the option of selecting among one or more different, including multiple different, inputs, e.g., various parameter values for the algorithm, as described above, such as distance from 3′ end, definition of overlap, t, etc. Computational systems that may be readily modified to become systems of the subject invention include those described in U.S. Pat. No. 6,251,588; the disclosure of which is herein incorporated by reference.

Utility

The above-described methods and devices programmed to practice the same may be used to identify normalization probe nucleic acids to be produced on surfaces of any of a variety of different substrates, including both flexible and rigid substrates, e.g., in the production of nucleic acid arrays. Materials of interest provide physical support for the deposited material and endure the conditions of the deposition process and of any subsequent treatment or handling or processing that may be encountered in the use of the particular array. The array substrate may take any of a variety of configurations ranging from simple to complex. Thus, the substrate could have generally planar form, as for example, a slide or plate configuration, such as a rectangular or square disc. In many embodiments, the substrate will be shaped generally as a rectangular solid, having a length in the range of about 4 mm to 200 mm, usually about 4 mm to 150 mm, more usually about 4 mm to 125 mm; a width in the range of about 4 mm to 200 mm, usually about 4 mm to 120 mm, and more usually about 4 mm to about 80 mm; and a thickness in the range of about 0.01 mm to about 5 mm, usually from about 0.1 mm to about 2 mm and more usually from about 0.2 mm to about 1 mm. However, larger or smaller substrates may be and can be used, particularly when such are cut after fabrication into smaller size substrates carrying a smaller total number of arrays 12. Substrates of other configurations and equivalent areas can be chosen. The configuration of the array may be selected according to manufacturing, handling, and use considerations.

The substrates may be fabricated from any of a variety of materials. In certain embodiments, such as for example where production of binding pair arrays for use in research and related applications is desired, the materials from which the substrate may be fabricated should ideally exhibit a low level of non-specific binding during hybridization events. In many situations, it will also be preferable to employ a material that is transparent to visible and/or UV light. For flexible substrates, materials of interest include: nylon, both modified and unmodified, nitrocellulose, polypropylene, and the like, where a nylon membrane, as well as derivatives thereof, may be particularly useful in this embodiment. For rigid substrates, specific materials of interest include: glass; fuse silica; silicon, plastics (for example polytetraflouroethylene, polypropylene, polystyrene, polycarbonate, and blends thereof, and the like); metals (for example, gold, platinum, and the like).

The substrate surface onto which the probe nucleic acid compositions or other moieties are deposited may be smooth or substantially planar, or have irregularities, such as depressions or elevations. The surface may be modified with one or more different layers of compounds that serve to modify the properties of the surface in a desirable manner. Such modification layers of interest include: inorganic and organic layers such as metals, metal oxides, polymers, small organic molecules and the like. Polymeric layers of interest include layers of: peptides, proteins, polynucleic acids or mimetics thereof (for example, peptide nucleic acids and the like); polysaccharides, phospholipids, polyurethanes, polyesters, polycarbonates, polyureas, polyamides, polyetheyleneamines, polyarylene sulfides, polysiloxanes, polyimides, polyacetates, and the like, where the polymers may be hetero- or homopolymeric, and may or may not have separate functional moieties attached thereto (for example, conjugated).

Arrays

Also provided by the subject invention are nucleic acid arrays of produced using the subject methods, as described above. The subject arrays include at least one probe, and typically a plurality of different probes of different sequence (e.g., at least about 10, usually at least about 50, such as at least about 100, 1000, 5000, 10,000 or more) immobilized on, e.g., covalently or non-covalently attached to, different and known locations on the substrate surface. A feature of the subject arrays is that at least one of the probes is a normalization probe having a sequence identified according to the present methods, where in many embodiments at least about 5, 10, or more of the probe sequences are normalization sequences identified by the subject methods. Each distinct nucleic acid sequence of the array is typically present as a composition of multiple copies of the polymer on the substrate surface, e.g. as a spot on the surface of the substrate. The number of distinct nucleic acid sequences, and hence spots or similar structures (i.e., array features), present on the array may vary, but is generally at least 2, usually at least 5 and more usually at least 10, where the number of different spots on the array may be as a high as 50, 100, 500, 1000, 10,000 or higher, depending on the intended use of the array. The spots of distinct nucleic acids present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In the subject arrays of nucleic acids, the nucleic acids may be covalently attached to the arrays at any point along the nucleic acid chain, but are generally attached at one of their termini, e.g., the 3′ or 5′ terminus.

A feature of the subject arrays is that they include one or more, usually a plurality of, normalization probes whose sequence as been selected according to the subject protocols. Because the sequences of the normalization probes on the arrays are selected according to the above protocols, the normalization probe sequences are ones that provide for high signal with little variation under a plurality of different differential gene expression protocols. For example, one or more of the normalization probe sequences on the array will provide performance that varies little, if any, between two or more different differential gene expression assays, i.e., it performs substantially similar under a plurality of different experimental conditions, e.g., as determined by exhibiting a log ratio across a plurality of different experimental sets that does not significantly vary from zero.

Utility of Arrays

The subject arrays find use in a variety of different applications, where such applications are generally analyte detection applications in which the presence of a particular analyte in a given sample is detected at least qualitatively, if not quantitatively. Protocols for carrying out such assays are well known to those of skill in the art and need not be described in great detail here. Generally, the sample suspected of comprising the analyte of interest is contacted with an array produced according to the subject methods under conditions sufficient for the analyte to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g. through use of a signal production system, e.g., an isotopic or fluorescent label present on the analyte, etc. The presence of the analyte in the sample is then deduced from the detection of binding complexes on the substrate surface.

Specific analyte detection applications of interest include hybridization assays in which the nucleic acid arrays of the subject invention are employed. In these assays, a sample of target nucleic acids is first prepared, where preparation may include labeling of the target nucleic acids with a label, e.g., a member of signal producing system. Where the arrays include “all-bases-all-layers” control probes, as described above, a collection of labeled control targets is typically included in the sample, where the collection may be made up of control targets that are all labeled with the same label or two or more sets that are distinguishably labeled with different labels, as described above. Following sample preparation, the sample is contacted with the array under hybridization conditions (e.g., stringent hybridization conditions), whereby complexes are formed between target nucleic acids that are complementary to probe sequences attached to the array surface. The presence of hybridized complexes is then detected. Specific hybridization assays of interest which may be practiced using the subject arrays include: gene discovery assays, differential gene expression analysis assays; nucleic acid sequencing assays; and the like. Patents and patent applications describing methods of using arrays in various applications include: 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

As such, in using an array made by the method of the present invention, the array will typically be exposed to a sample (for example, a fluorescently labeled analyte, e.g., protein containing sample) and the array then read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Palo Alto, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

Kits

Kits for use in analyte detection assays are also provided. The kits at least include the arrays of the invention, as described above. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kits may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the array assay devices for carrying out an array based assay. Such kits also typically include instructions for use in practicing array based assays.

Kits for use in connection with the normalization probe design protocols of the subject invention may also be provided. Such kits preferably include at least a computer readable medium including programming as discussed above and instructions. The instructions may include installation or setup directions. The instructions may include directions for use of the invention.

Providing software and instructions as a kit may serve a number of purposes. The combinations may be packaged and purchased as a means of upgrading an existing fabrication device. Alternatively, the combination may be provided in connection with a new device for fabricating arrays, in which the software may be preloaded on the same. In which case, the instructions will serve as a reference manual (or a part thereof) and the computer readable medium as a backup copy to the preloaded utility.

The instructions of the above-described kits are generally recorded on a suitable recording medium. For example, the instructions may be printed on a substrate, such as paper or plastic, etc. As such, the instructions may be present in the kits as a package insert, in the labeling of the container of the kit or components thereof (i.e. associated with the packaging or sub packaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or World Wide Web. Some form of access security or identification protocol may be used to limit access to those entitled to use the subject invention. As with the instructions, the means for obtaining the instructions and/or programming is generally recorded on a suitable recording medium.

The following examples are offered by way of illustration and not by way of limitation.

### EXPERIMENTAL

A. Candidate Probe Selection:

Ten 60-mer probe sequences were designed for each of approximately 18,232 human sequences using designed using Agilent's probe design software package (Agilent Technologies, Palo Alto Calif.). This software package selects probes according to the following criteria:
Distance from the 3′-end of the mRNA. This criterion affects both predominantly sensitivity. Probes are generally chosen within a defined distance (bases) from the polyadenylation site of the mRNA. This is because the nucleic acid target synthesis is usually primed from this site, and the efficiency of target production usually falls off with distance from the primer.
Base composition of the probe sequence. This criterion affects both sensitivity and specificity. Sequences that are abnormally GC rich or poor are avoided, as are long runs of a single base.
Homology of the probe sequence to other sequences from the same organism. This criterion mainly affects specificity. Sequences with a high potential to hybridize to more than one mRNA from a given organism are avoided. Cross-hybridization potential is estimated via thermodynamic scoring of the output of BLAST, a standard bioinformatics application used to detect sequence homology.
   B. Hybridization of Candidate Probes:

Candidate 60-mer probes specified by Aglient's probe design algorithms were laid out onto 22.5 K array designs and the microarrays were printed using Agilent's SurePrint in situ oligonucleotide synthesis process. These microarrays were hybridized to 10 different tissue/cell line combinations (4 replicates per sample pair): one self-vs-self and 9 sample pairs chosen to maximize the number of probes throwing log ratio values within the experimental set. The sample set used consisted of the following cRNA target pairs: 1) Brain (red) and Placenta (green), 2) HeLa (red) and Clontech Reference sample (green), 3) HeLa (red) and HeLa (green), 4) K-562 (red) and Clontech Reference sample (green), 5) K-562 (red) and MG63 (green), 6) Lung (red) and Liver (green), 7) Lung (red) and Placenta (green), 8) Placenta (red) and Clontech Reference sample (green), 9) Spleen (red) and HeLa (green), 10) Stratagene Reference sample (red) and Clontech Reference sample (green). The labeled tissue-derived samples were prepared from commercially available poly-adenylated RNA or total RNA (Ambion, Inc., Woodlands, Tex., BD Clontech, Inc., Palo Alto, Calif., and Stratagene, Inc., La Jolla, Calif.) using Agilent's linear amplification kit. Hybridization, washing and scanning were performed according to procedures described in Agilent's Microarray User Manual.

After scanning, the feature data were extracted using Agilent's Feature Extraction software (version A.7.1.1) and the feature data was assembled into Microsoft Access databases using Agilent's Kiwi II application.

C. Apply CAST Clustering Algorithms.

The strategy of using clustering analysis for experimental probe validation is based assumption that probes that hybridize to a single target will behave similarly in gene expression experiments, both within a single experimental pair and across multiple experimental pairs. Disparate log ratio values for probes designed to a single target may be caused by a variety of factors that include non-specific hybridization of additional target(s), probe secondary structure or other factors that limit hybridization efficiency, misannotation of target structure (eg: intron/exon boundaries) and labeling biases. Most, if not all, of these factors cannot be accurately predicted using “in silico” methods. Clustering techniques have been used in the analysis of gene expression data to identify genes that are co-regulated. For probe validation, we used CAST (Cluster Affinity Search Technique) clustering algorithms (Ben-Dor A., et al (1999), J. Comput. Biol. 6, 281-297) to identify co-regulated probes from the candidate probes designed to target a single gene. CAST is a non-greedy clustering algorithm that constructs clusters by preserving a high intra-cluster similarity at all stages. This level of similarity is determined by an input parameter τ. These algorithms have several advantages over other clustering algorithms for this application: they form a non-hierarchical clustering (i.e.: the clusters are unrelated and cluster boundaries are determined by the algorithm) and they do not assume a given number of clusters (i.e.: the number of clusters is determined by the algorithm instead of being a constant given as an input parameter).

Cluster memberships for candidate probes designed to human genes were identified using the Agilent software package “OC Analysis”. The application performs the following steps:
Generation of expression matrix. Replicate log ratio values for a given sample pair are combined using error-weighted averaging. The combined log ratio data for candidate probes designed to target a single gene are used to populate an expression matrix I where Iᵢⱼ is the measured expression level of probe i in experiment (condition) j. Only those probes that exceed a user-specified signal threshold on any of the combined arrays are included in the expression matrix. The size of the expression matrix is dependent on the similarity measure used in the clustering algorithm. For example, the significance of the Pearson's correlation coefficient depends on the number of experiments, and expression matrix consisting of at least 8 experiments is ideal. Performance of the clustering algorithm does not depend on the number of probes, since it assigns probes to clusters based on the affinity to cluster, however, the number of probes should be high enough to be representative of all possible probes for the input sequence. Thus, the clustering algorithm is able to work with a matrix with some null entries.
Calculation of a similarity matrix S. In this matrix the entry Sᵢⱼ represent the similarity of the expression pattern for probes i and j. The similarity measure used by CAST for this step is independent of the clustering mechanism. Specific examples are the Pearson's correlation coefficient and Kendall's rank correlation.
Clustering of probes using CAST. The CAST clustering algorithms partition the probes into groups based on similar expression patterns. The input to the algorithm is a pair (S, □) where S is a n-by-n similarity matrix and τis user-specified affinity threshold that determines what affinity level is considered significant. The algorithm constructs clusters incrementally and uses average similarity (affinity) between unassigned vertices and the current cluster to make its next decision to add or remove elements from groups. The clusters are “stable” when the average similarity exceeds the affinity threshold (τ). The OC Analysis application allows input of up to 5 τvalues and iteratively performs the cluster analysis at decreasing affinity thresholds until a cluster of a user-defined minimum size is formed. Cluster membership is assigned for each cluster and a cluster size and a cluster quality score is calculated. The quality score of a cluster is a measure of the likelihood of such a cluster occurring if data from unrelated probes from the data set were clustered. High probability clusters (i.e.: those where the data clusters much more tightly than would be expected from randomly selected data) are given high scores.
   D. Use Clustering Metrics to Identify Probes in the “Best Cluster”.

The OC Analysis application identifies the “best cluster” based on the affinity threshold used and the size of the cluster formed. For those targets where the candidate probes partitioned into multiple groups, the “representative cluster” was chosen as the cluster formed at the highest τ that allowed formation of cluster at least 50% larger than the next largest cluster and a minimum cluster size of 4 elements. These criteria were chosen so that “representative clusters” comprise a majority of the probes tested for a given target sequence. For the 15,032 genes where acceptable clusters were identified, only 4,315 (29%) showed similar gene expression patterns for 10 of the 10 candidate probes tested; the remaining 71% had at least one candidate probe that showed distinct patterns.

The specific algorithms used in this application, the software code and a procedure describing the use of this application for empirical probe selection for Agilent Catalog Microarray Products are described in U.S. patent application Ser. No. ______ (Attorney Docket No. 10021251-1) the disclosure of which is herein incorporated by reference.

Selection of Normalization Probes. The initial selection criteria selects candidate probes showing no significant log ratio changes across the experimental set. Confidence intervals (99%) were calculated around replicate mean log ratios values (n=4). Probes were selected when the mean log ratio values were not significantly different from 0 for each of the 10 experimental samples. Only 254 probes, each designed to a different target, showed no significant differential expression across the highly divergent experimental set. Given that the initial probe set comprised 182319 probes for 18232 human sequences, this result suggests that “housekeeping genes” (genes that are “universally” expressed at the same levels in all tissues) are extremely rare. There were no cases where multiple probes to a given target showed no significant differential expression across the experimental set (which would be expected if the apparent lack of differential expression reflected constant relative target levels).

Probes were further selected as normalization probes if they met the following criteria: 1) they were derived from a target sequence where an acceptable candidate probe cluster was identified, and 2) the probe was not included in the acceptable cluster. This selection was performed to select against probes that did not show differential expression simply because the complementary target was not represented in the sample set. In fact, the distribution of signal intensities for the probes that met these criteria was higher than the signal intensities for the probes that did not (data not shown). 104 probes were selected from the initial set as normalization probes using these criteria.

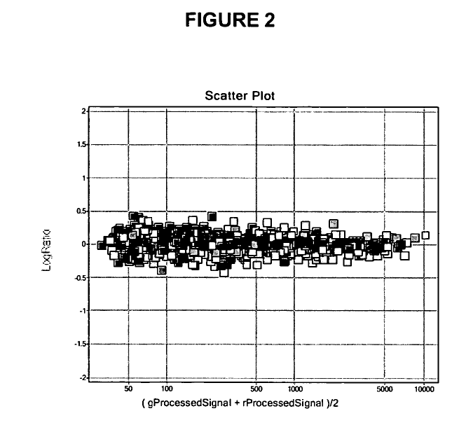

FIG. 2 signal intensities and log ratio values for the 10 experimental sample pairs (colored by experimental sample pair) for the normalization probes selected using this method. The signal intensities of these probes span the dynamic range of the microarray platform, a useful if not essential characteristic for robust normalization performance.

It is evident from the above results and discussion that a new and useful method of designing normalization probes for use on nucleic acid microarrays is provided by the subject invention. Normalization probes identified according to the subject methods exhibit high signals with little variation across a large number of divergent nucleic acid samples, and therefore are particularly suited for use as normalization probes. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of identifying a sequence of a nucleic acid that is suitable for use as a substrate surface immobilized normalization probe, said method comprising:
(a) identifying a plurality of candidate probe sequences for a target nucleic acid based on at least one selection criterion;
(b) empirically evaluating each of said candidate probe sequences under a plurality of different experimental sets to obtain a collection of empirical data values for each of said candidate nucleic acid probe sequences for each of said plurality of different experimental sets;
(c) clustering said candidate probe sequences into one or more groups of candidate probe sequences based on each candidate probe sequence's collection of empirical data values, wherein each of said one or more groups exhibits substantially the same performance across said plurality of experimental sets;
(d) evaluating any remaining non-clustering probes for candidate probe sequences that satisfy a signal intensity threshold and exhibit substantially no variation in signal under said plurality of different experimental sets to identify any candidate probe sequences of said plurality that are suitable for use as a substrate surface immobilized normalization probe.

2. The method according to claim 1, wherein said at least one selection criterion employed in said identifying step (a) is chosen from:
(i) proximity to the 3′ end of said target nucleic acid's corresponding mRNA transcript;
(ii) base composition; and
(iii) lack of homology to other expressed sequences of said target nucleic acid's organism.

3. The method according to claim 2, wherein all three of said selection criteria (i), (ii) and (iii) are employed is said identifying step (a).

4. The method according to claim 3, wherein said identifying step (a) is further characterized by employing parameters that minimize the number of identified candidate probe sequences that overlap with each other.

5. The method according to claim 1, wherein said empirically evaluating step (b) comprises for each member of said plurality of different experimental conditions:
(i) providing an array of candidate nucleic acid probes immobilized on a surface of a solid support, wherein said array includes a substrate surface immobilized nucleic acid candidate probe for each of said identified candidate probe sequences; and
(ii) subjecting said array to said member of said plurality of different experimental sets.

6. The method according to claim 5, wherein each member of said plurality of different experimental conditions is a different tissue/cell line differential gene expression assay.

7. The method according to claim 1, said clustering step (c) comprises:
(i) obtaining an expression vector for each of said candidate probe sequences using said candidate sequence's collection of empirical data values;
(ii) deriving a similarity matrix for the set of said candidate probe sequences from said candidate probe sequences' expression vectors; and
(iii) grouping said candidate probe sequences based on their derived similarity.

8. The method according to claim 7, wherein those candidate probes that have substantially similar expression patterns are grouped together.

9. The method according to claim 1, wherein the clustering step employs an affinity threshold or another stringency controlling parameter.

10. The method according to claim 1, wherein a candidate probe sequence is considered to exhibit substantially no variation in signal under said plurality of different experimental sets if its log ratio is not significantly different than zero accorss said plurality of different experimental sets.

11. The method according to claim 10, wherein said log ratio is between about 0.5 and −0.5.

12. The method according to claim 1, wherein said purality of different experimental sets is at least 2.

13. The method according to claim 12, wherein if no non-clustering probes are present after said clustering step (c), said evaluating step (d) is not performed.

14. The method according to claim 1, wherein at least some of said steps are carried out by a computational analysis system.

15. A computer-readable medium having recorded thereon a program that identifies a sequence of a nucleic acid that is suitable for use as a substrate surface immobilized normalization probe according to the method of claim 1.

16. A computational analysis system comprising a computer-readable medium according to claim 15.

17. A method of producing a nucleic acid array, said method comprising:
producing at least two different probe nucleic acids immobilized on a surface of a solid support, wherein at least one of said at least two different probe nucleic acids is a normalization probe that has a sequence of nucleotides identified according to the method of claim 1.

18. The method according to claim 17, wherein said at least two different probe nucleic acids are produced on said surface of said solid support by synthesizing said probe nucleic acids on said surface.

19. The method according to claim 17, wherein said at least two different probe nucleic acids are produced on said surface of said solid support by depositing said at least two different probe nucleic acids onto said surface of said solid support.

20. A nucleic acid array produced according to the method of claim 17.

21. A method of detecting the presence of a nucleic acid analyte in a sample, said method comprising:
(a) contacting a nucleic acid array according to claim 20 having a nucleic acid probe that specifically binds to said nucleic acid analyte with a sample suspected of comprising said analyte under conditions sufficient for binding of said analyte to said nucleic acid ligand on said array to occur; and
(b) detecting the presence of binding complexes on the surface of said array to detect the presence of said analyte in said sample.

22. A method comprising transmitting a result of a reading of an array obtained according to the method of claim 20 from a first location to a second location.

23. The method according to claim 22, wherein said second location is a remote location.

24. A method comprising receiving a transmitted result of a reading of an array obtained according to the method claim 20.

25. A kit for identifying a sequence of a nucleic acid that is suitable for use as a substrate surface immobilized normalization probe, said kit comprising:
(a) an algorithm that identifies a sequence of a nucleic acid that is suitable for use as a substrate surface immobilized normalization probe according to the method according to claim 1, wherein said algorithm is present on a computer readable medium; and
(b) instructions for using said algorithm to identify said sequence of a nucleic acid that is suitable for use as a substrate surface immobilized normalization probe .
